(19) United States
(12) Patent Application Publication
(10) Pub. No.: US 2022/0168109 A1
(43) Pub. Date: Jun. 2, 2022

(54) PROSTHETIC IMPLANT

(71) Applicant: BIORL S.R.L., Melzo (Milano) (IT)

(72) Inventors: Gérard GIORDANO, Melzo (Milano) (IT); Redouane SELMOUNE, Bollate (Milano) (IT)

(21) Appl. No.: 17/369,053

(22) Filed: Jul. 7, 2021

(30) Foreign Application Priority Data
Nov. 27, 2020 (IT) 102020000028751

(51) Int. Cl. A61F 2/34

(52) U.S. Cl. CPC A61F 2/34; A61F 2002/30677

(57) ABSTRACT

A prosthetic implant shaped as a portion of a human skeletal structure and including a contact and delivery portion shaped as a porous structure, in particular as a trabecular type structure, suitable for receiving a medical substance and configured to be arranged in contact with a bone of a patient during an operating configuration of the prosthetic implant and a support portion, coupled to the contact and delivery portion, including a plurality of ducts each having at least one inlet section, made at an operative part of the prosthetic implant that can be accessed from the outside by the surgeon in the operating configuration, and at least one outlet section which opens into the contact and delivery portion. The contact and delivery portion is configured to deliver the medical substance evenly onto the bone when the medical substance is injected through at least one duct of said plurality of ducts during the operating configuration.

This application claims priority to Italian Patent Application 102020000028751 filed Nov. 27, 2020, the entirety of which is incorporated by reference herein.

The present invention relates to a prosthetic implant that is widely used in the medical field.

As it is well known, during the life of a person, the human body can be subjected to various traumas, inflammatory, infectious, tumour, autoimmune and degenerative diseases (such as arthrosis) that can damage and deteriorate certain bones. These attacks affecting the joints lead to a reduction in the quality of life and skills of the person, in particular, the functions of adapting to the environment (locomotion, gripping and the like). In older people, such joint attacks can also lead to a decrease in life expectancy.

Until now these diseases have been medically treated in a conservative way. However, in some cases the treatments are insufficient and therefore the patient needs to be treated by inserting a prosthetic implant.

Prosthetic implants can be fixed to the patient's bone using cement, such as a polymethyl methacrylate (PMMA) acrylic polymer, or without cement. In the latter case, the primary anchorage of the prosthetic implant is ensured by a mechanical press-fit effect to which, in the months following the installation of the implant in the patient, a secondary-biological anchorage obtained by the osseointegration of the prosthetic implant with the bone interface is added. The process of osseointegration is therefore crucial for the prosthetic implant correct functioning and constitutes the limit of the current “mechanistic” conception of the anchorage of joint implants.

In other words, when a person develops, as time goes by, serious damage at a joint (e.g. hip, shoulder and the like) resulting in a fractured or deteriorated bone, it is necessary to completely replace the injured part with an internal medical prosthesis which must be fixed within the patient, as mentioned above, by a primary and a secondary-biological fixation.

It is also known that, following the installation of a prosthetic implant, it is possible that, in the contact area between the bone and the implant itself, the trabeculae of the bone are subject to high compressive loads, such as the body weight. In this situation, the trabeculae of the bone become very compact, resulting in a bone that is defined as “sclerotic”, i.e. a bone that does not have a proper vascularisation. This leads to bone shrinkage (and therefore incorrect secondary fixation of the prosthetic implant), infections and similar problems.

Currently, in order to overcome this problem, for example in hip prostheses, and more precisely when implanting the cup, small holes are made in the bone after milling, so that the bone bleeds and osteoblasts come into contact with the prosthetic implant, promoting its osseointegration.

Disadvantageously, this technique has some limitations in that the prosthetic implant does not always osseointegrate properly, making it necessary to revise the implant.

Known prosthetic implants therefore have disadvantages related to their accessibility by the surgeon once they have been installed.

In particular, if the installed implant fails to osseointegrate into the patient's skeletal structure, it causes bone loss and loosening of the prosthetic implant itself. In such a situation, it is necessary to operate by applying medication and/or by revising the implant. Since the area of the prosthetic implant on which the surgeon needs to operate is the area in contact with the patient's skeletal structure, it can be accessed by the surgeon through highly invasive operations on the patient.

The technical task of the present invention is therefore to make available a prosthetic implant capable of overcoming the drawbacks of the prior art.

The object of the present invention is therefore to make available a prosthetic implant which allows medical substances to be administered in areas which cannot be directly accessed, or which can no longer be accessed, by the surgeon, without having to perform highly invasive operations on the patient.

A further object of the present invention is therefore to make available a prosthetic implant capable of promoting the osseointegration and the correct vascularisation of the bone tissue.

A further object of the present invention is therefore to make available an easy-to-install, effective and reliable prosthetic implant.

The technical task specified, and the objects specified are substantially achieved by a prosthetic implant comprising the technical features set forth in one or more of the appended claims. The dependent claims correspond to possible embodiments of the invention.

In particular, the specified technical task and the specified objects are achieved by a prosthetic implant shaped as a portion of a human skeletal structure according to the present invention. The prosthetic implant advantageously comprises a contact and delivery portion shaped as a porous structure, in particular as a trabecular structure, suitable for receiving a medical substance and configured to be arranged in contact with a patient's bone during an operating configuration of the prosthetic implant. The prosthetic implant also advantageously comprises a support portion, coupled to the contact and delivery portion and comprising a plurality of ducts each having at least one inlet section, made in an operative part of the prosthetic implant that can be accessed from the outside by the surgeon in the operating configuration, and at least one outlet section which opens into the contact and delivery portion.

Thanks to the present invention, the contact and delivery portion is configured to deliver the medical substance evenly onto the bone when the medical substance is injected through at least one duct of the plurality of ducts during the operating configuration.

In other words, the prosthetic implant, which is the subject of the present invention, allows the surgeon to inject a medical substance into the inlet sections of the ducts of the support portion so that the substance is pressurised and flows into the vicinity of the contact and delivery portion. In this situation, the medical substance is distributed through the porous structure of the contact and delivery portion to the bone in contact therewith. The delivery therefore occurs gradually and evenly through the porosities of the structure.

Advantageously, the presence and shape of the ducts allow the medical substance, such as a drug, to be delivered to an area of the implant which cannot be accessed by the surgeon once the prosthetic implant has been installed.

Advantageously, the presence of the ducts facilitates the administration of drugs and/or substances for the maintenance of the prosthesis with no need to remove the prosthesis entirely or to perform particularly invasive surgery on the patient.

Further characteristics and advantages of the present invention will become clearer from the indicative and therefore non-limiting description of an embodiment of a prosthetic implant.

Such description will be made herein below with reference to the accompanying drawings, provided for indicative purposes only and therefore not limiting, wherein:

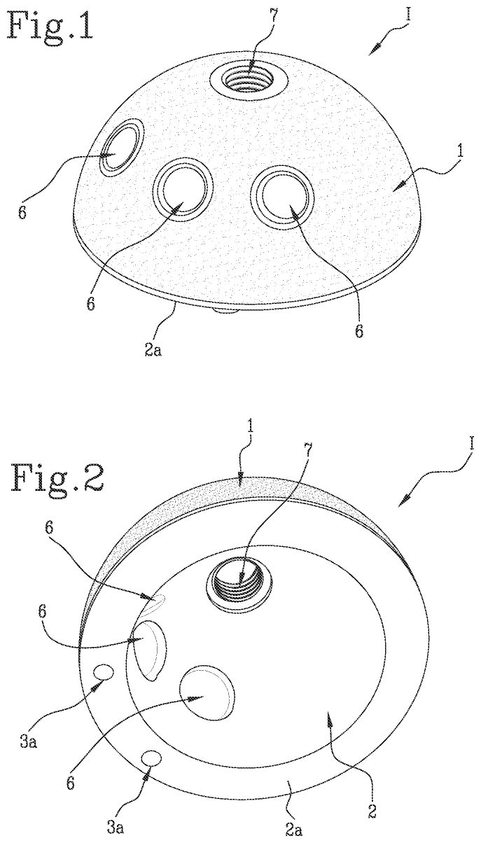

FIG. 1 shows a schematic perspective view of an embodiment of a prosthetic implant according to the present invention;

FIG. 2 shows a schematic view from below of the embodiment of the implant of FIG. 1;

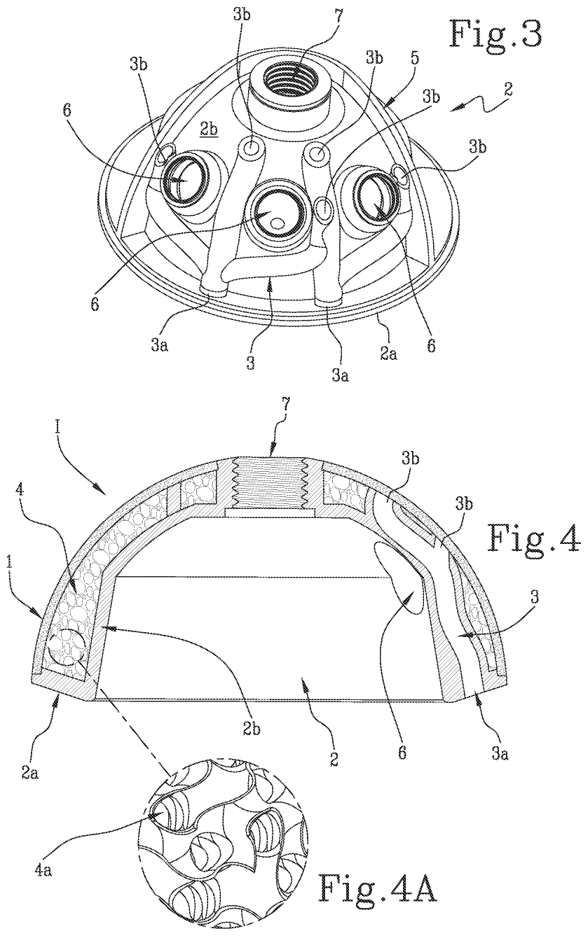

FIG. 3 shows a schematic perspective view of an inner portion of the embodiment of the prosthetic implant of FIG. 1;

FIGS. 4 and 4A show a schematic sectional view of the implant and a detail of FIG. 4 respectively.

With reference to the accompanying figures, “I” has generally been used to denote a prosthetic implant shaped like a portion of a human skeletal structure, such as a tibial base, cup and the like.

The prosthetic implant “I” comprises a contact and delivery portion 1 shaped as a porous structure, in particular as a trabecular type structure, suitable for receiving a medical substance. The medical substance can be selected from, but is not limited to, an antibiotic, a PRP (Platelet-Rich Plasma) substance, high viscosity cement and drugs for osseointegration.

The contact and delivery portion 1 is configured to be arranged in contact with a bone of a patient, not shown in the accompanying figures, during an operating configuration of the prosthetic implant “I” so as to deliver on said contacted bone the received medical substance, as will be described in detail below.

The prosthetic implant “I”, subject of the present invention, further comprises a support portion 2 coupled to the contact and delivery portion 1 and, preferably, comprising an operative part 2a of the prosthetic implant “I” which can be accessed from the outside by the surgeon in the operating configuration.

The term operating configuration refers to a configuration in which the prosthetic implant “I” occupies its physiological position within the patient. In this situation, the contact and delivery portion 1 is in contact with the patient's bone and cannot therefore be accessed by the surgeon from the outside for direct supply of the medical substance, while, as mentioned above, the operative part 2a can be accessed by, or exposed to, the surgeon.

The support portion 2 further comprises a plurality of ducts 3 each having at least one inlet section 3a, made on the operative portion 2a, and at least one outlet section 3b which opens into the contact and delivery portion 1.

As it will be described in detail below, each duct 3 of the plurality of ducts 3 is configured to allow supplying the medical substance to the contact and delivery portion 1 in such a way that the latter irradiates the bone, with which it is in contact, with that substance. When the medical substance is injected through at least one duct 3 of the plurality of ducts 3 during the operating configuration, the contact and delivery portion 1 is in fact configured to deliver the medical substance evenly onto the bone.

In other words, in order for the contact and delivery portion 1 to deliver a medical substance onto the bone in contact therewith, such medical substance is injected through the inlet section 3a within the ducts 3 of the plurality of ducts 3 so as to flow into the contact and delivery portion 1 itself. In such a situation, the medical substance passes through the ducts 3 to the trabecular structure of the contact and delivery portion 1 where it is gradually delivered towards the patient's bone.

In a possible embodiment, not shown, each duct 3 of the plurality of ducts is made as a single pipe having a single inlet section 3a, located on the operative part 2a of the prosthetic implant “I”, and a single outlet section 3b.

Alternatively, as shown in the embodiment of FIG. 3, each duct 3 may be of a branched type so as to have one inlet section 3a and several outlet sections 3b to ensure spraying more evenly the medical substance within the entire trabecular structure of the contact and delivery portion 1.

According to a further embodiment, the ducts 3 may be interconnected between each other so as to be placed in fluid communication with each other to define a network of different paths within the support portion 2.

Preferably, the ducts 3 have a diameter between 0.5 mm and 4.5 mm, even more preferably of 2.5 mm. These dimensions may vary depending on the size and type of prosthetic implant “I” that can be installed in the patient.

Advantageously, the presence of ducts 3 allows a medical substance to be supplied to areas that cannot be directly accessed by the surgeon when the prosthetic implant “I” is in situ. In this way, problems of osseointegration, infection and fixation of the prosthetic implant “I” due to areas which cannot be accessed to, are substantially eliminated.

Advantageously, the possibility of administering a medical substance even into areas that cannot be accessed by the surgeon avoids invasive interventions on the patient as it avoids, for example, having to remove the entire prosthetic implant “I” for a targeted administration of the medical substance into the area in contact with the patient's bone.

In use, therefore, when the installation of the prosthetic implant “I” takes place, the surgeon places the latter in the operating configuration, i.e. in its physiological position inside the patient in such a way that the contact and delivery portion 1 is in direct contact with the bone with which the prosthetic implant “I” is to be integrated.

Once the “I” prosthetic implant has been positioned, as described above, the contact and delivery portion 1 can therefore no longer be accessed from the outside by the surgeon.

In such a situation, in order to allow supplying the medical substance to the contact and delivery portion 1, and thus to administer such substance to the patient's bone, the surgeon injects, by means of an injection device, of a known and not illustrated type, the medical substance within the at least one inlet section 3a of each duct 3 located on the operative part 2a which can be accessed from the outside, i.e. exposed towards the surgeon.

Preferably, the injection device is made in the form of a syringe having a needle which is at least partially inserted into each inlet section 3a of the ducts 3 of the plurality of ducts 3 to inject the medical substance.

Following the injection operation, the medical substance is pressurised within the ducts 3 and is therefore able to flow within the ducts 3 to the at least one outlet section 3b to flow into the contact and delivery portion 1.

Preferably, since the structure of the contact and delivery portion 1 is highly porous, to ensure retention of the medical substance within such portion, the support portion 2 is coupled to the contact and delivery portion 1 so as to define a containment wall 2b of the medical substance within the porous structure.

In other words, the support portion 2 is coupled to the contact and delivery portion 1 in such a way as to delimit a bottom closure of the porosities of the contact and delivery portion 1 to ensure containment of the medical substance within the contact and delivery portion 1 itself while preventing leakage to areas that do not contact the bone.

In such a situation, the medical substance is contained within the porous trabecular structure of the contact and delivery portion 1 which carries the medical substance and distributes it evenly on the bone.

In other words, a kind of “fluid-tight chamber” is created whereby the pressure pushes the medical substance towards the bone.

In particular, the highly porous structure of the contact and delivery portion 1 acts as an anastomotic capillary network, allowing to deliver a weighted amount of the medical substance over the entire surface contacting the bone while maintaining a reserve load within the structure itself.

In the preferred embodiment, the prosthetic implant “I” is made of titanium alloy, particularly of an alloy comprising Titanium, Aluminium and Vanadium according to the Ti₆Al₄V formula.

Alternatively, the prosthetic implant “I” can be made of an alloy comprising Cobalt, Chromium and Molybdenum.

Preferably, the prosthetic “I” implant is made using an additive manufacturing technique.

Advantageously, making the prosthetic implant “I”, and in particular the contact and delivery portion 1, by means of the aforementioned additive manufacturing techniques, makes it possible to create a highly porous and rough structure capable of promoting and speeding up considerably the osseointegration of the bone contacting the prosthetic implant “I”.

Still more preferably, the contact and delivery portion 1 and the support portion 2 are made in a single piece by means of an additive manufacturing technique, e.g. by Electron Beam Melting (EBM) or Direct Metal Laser Melting (DMLS).

Advantageously, making the contact and delivery portion 1 and the support portion 2 in a single piece avoids material export processing for making the porous structure and/or the ducts 3. Such processing could result in residues that would be harmful to the patient's health if they became embedded in the porous structure of the contact and delivery portion 1.

With particular reference to the embodiment shown in the appended figures, a prosthetic implant “I” according to the present invention made in the form of a cup is shown.

With particular reference to FIG. 1, in the cup, the contact and delivery portion 1 defines an outer hemispherical shell capable of engaging a patient's acetabulum during the operating configuration.

In such a situation, the outer hemispherical shell, thanks to its highly porous trabecular structure, allows to deliver the medical substance to the acetabulum, i.e. the bone cavity adapted to receive the cup, as well as to promote the osseointegration of the prosthetic implant “I” with the acetabulum itself.

Preferably the outer hemispherical shell has a thickness having a value between 0.8 mm and 6 mm, still more preferably of 2 mm.

Referring now to FIGS. 2 and 3, in the cup, the support portion 2 defines an inner hemispherical shell coupled internally to the outer hemispherical shell.

In the embodiment illustrated in the attached figures, the inner hemispherical shell further comprises a containment wall 2b shaped to contain the medical substance within the contact and delivery portion 1 when injected in the operating configuration.

As shown in the embodiment of FIGS. 2 and 3, the containment wall 2b has a substantially hemispherical shape adapted to be fitted within the outer hemispherical shell in such a way that, the medical substance, delivered from the ducts 3 into the porous trabecular structure of the contact and delivery portion 1, is retained within said portion 1 preventing it from escaping towards the inside of the cup.

In a further embodiment, the containment wall 2b can be made by using bone cement so as to partially obstruct the porosity of the trabecular structure of the contact and delivery portion 1, avoiding, in this case also, a leakage of the medical substance towards the inside of the cup.

As shown in FIG. 2, the inner hemispherical shell is further provided with a bottom surface 2a shaped as a circular crown and defining the operative part 2a of the prosthetic implant “I” that can be accessed from the outside by the surgeon.

In the preferred embodiment, the inner hemispherical shell thus consists of the containment wall 2b, which is dome-shaped to fit within the outer hemispherical shell, and the bottom surface 2a, which is circular-shaped and that can be accessed by the surgeon when the “I” prosthetic implant is in the operating configuration, i.e. placed in situ in the patient.

In this embodiment, the ducts 3 extend from the bottom surface 2a and branch out following the curvature of the dome defined by the containment wall 2b.

As shown in FIG. 4, the inner hemispherical shell further comprises at least one lightening portion 4 facing the outer hemispherical shell so as to be interposed between the outer hemispherical shell and the containment wall 2b.

As shown in FIG. 4A, the lightening portion 4 has a structure provided with a plurality of interconnected cavities 4a, preferably defining a gyroid type structure.

Preferably, the thickness of the lightening portion 4 has an average value between 0.8 mm and 12 mm, still more preferably 4 mm, while the diameter of the ducts 3 has a value between 0.5 mm and 4.5, even more preferably of 2 mm.

In the preferred embodiment, the inner hemispherical shell has multiple lightening portions 4 and preferably each duct 3 is at least partially, and more preferably totally, incorporated into the structure of the lightening portions 4.

Advantageously, the presence of one or more lightening portions 4 makes it possible to create, in cooperation with the trabecular structure of the outer hemispherical shell, a highly porous network capable of promoting a lightening of the inner structure of the cup.

Advantageously, the presence of one or more lightening portions 4 promotes even and gradual delivery of the medical substance injected by the surgeon through the ducts 3.

In the preferred embodiment, the inner hemispherical shell further comprises one or more ribs 5 extending from the containment wall 2b to the outer hemispherical shell so as to divide the at least one lightening portion 4 into two or more sectors each of which is associated with at least one outlet section 3b.

In the embodiment shown in FIG. 3, the inner hemispherical shell comprises a single substantially “U-shaped” rib 5 arranged such that the curvature of the “U” lies near the top of the cup. In such an embodiment, the rib 5 subdivides the lightening portions 4 into a first and a second sector in such a way that the outlet sections 3b of the ducts 3 are all associated with the first of the two sectors while the other is free of them. In such a situation, when the medical substance is delivered from the ducts 3, it is mainly conveyed and distributed in the first sector in such a way that in the portion of the outer hemispherical shell above the first sector, there is a higher concentration of the medical substance than in the portion above the second sector.

Advantageously, the presence of one or more ribs 5 allows to manage the distribution and delivery of the medical substance in different ways as necessary.

Advantageously, the presence of one or more ribs 5 makes the cup more robust and mechanically resistant.

In order to avoid bone inflammation, infection and/or regression following the installation of the “I” prosthetic implant, the surgeon needs to administer medical substances, such as cement for implant fixation and/or drugs for osseointegration, in the contact zone between the “I” prosthetic implant and the bone, which he cannot access directly.

In use, when installing the cup, the surgeon positions the cup so that the outer hemispherical shell is in contact with the patient's acetabulum. In such a situation, the outer hemispherical shell cannot be accessed by the surgeon, as it faces the patient's acetabulum, while the bottom surface 2a of the inner hemispherical shell can be accessed by the surgeon. In order to supply the medical substance also into the non-accessible area, the surgeon injects the substance through the inlet sections 3a of the ducts 3 by introducing an injection device in each inlet section 3a.

In this situation, the medical substance flows into the ducts 3 made on the inner hemispherical shell and out of each outlet section 3b of the ducts 3. The medical substance, once flown out of the ducts 3, is distributed within the porosity of the outer hemispherical shell with no risk that a part of it falls into the cup and is wasted. As shown in FIG. 4, the inner hemispherical shell, and more precisely the containment wall 2b, prevents the medical substance exiting the ducts 3 from falling towards the inside of the cup, thus retaining the medical substance itself. Therefore, the medical substance exiting the ducts 3 is constrained to be distributed within the trabecular structure of the outer hemispherical shell (and possibly also within the structure provided with cavities 4a of the lightening portions 4) in such a way that part of the medical substance is delivered immediately to the patient's acetabulum while a part remains stored in the trabecular structure and lightening portions 4 so that it is gradually delivered at a later time.

As shown in FIG. 3, the cup also comprises at least three through-holes 6 extending between the outer hemispherical shell and the inner hemispherical shell. Each through-hole 6 is configured to accommodate a means for fixing the cup to the acetabulum.

Preferably, the fixation means that can be inserted into each through-hole 6 can be made in the form of acetabular screws.

In the embodiment shown in the attached figures, such through-holes 6 are made in the first sector defined by the rib 5 in such a way that the ducts 3 extend around them.

The cup also includes an additional hole 7 placed at the top of the cup and configured to receive an instrument adapted to install it within the patient.

Preferably, the further hole 7 is threaded and also extends between the hemispherical outer shell and the hemispherical inner shell (FIG. 4).

The system “I” preferably further comprises at least one plug, not illustrated in the accompanying figures, configured to occlude the at least one inlet section 3a of the ducts 3 once the medical substance has been administered, so that the substance does not accidentally flow out after being injected.

It is also an object of the present invention to provide a kit for administering a medical substance in a contact zone between a prosthetic implant “I” and a bone of a skeletal structure of a patient. Such a kit comprises a prosthetic implant “I” according to what above described and installable in contact with a bone of a patient during an operating configuration and at least one injection device. Such an injection device contains a medical substance adapted to be injected through at least one duct 3 of the plurality of ducts 3 during the operating configuration.

Advantageously, the administration kit allows to speed up and make the installation of the prosthetic implant “I” more reliable because, attached to it, the injection device with the correct shape and size for such prosthetic implant “I” is provided.

The present invention achieves the intended objects by eliminating the drawbacks of the known art.

The prosthetic implant “I” allows medical substances to be applied in areas that cannot be accessed directly by the surgeon when the prosthetic implant “I” is in situ.

The prosthetic implant “I” avoids invasive surgery on the patient.

The conformation of the contact and delivery portion 1 allows the medical substance to be evenly distributed over the patient's bone in contact with it.

The conformation of the contact and delivery portion 1 promotes the osseointegration of the prosthetic implant “I” with the bone.

The possibility of making the “I” prosthetic implant using additive manufacturing techniques allows to obtain trabecular or gyroid structures that are highly porous and therefore able to overall lighten the “I” prosthetic implant.

1. A prosthetic implant shaped as a portion of a human skeletal structure and comprising:
a contact and delivery portion shaped as a porous structure, in particular as a trabecular structure, suitable for receiving a medical substance and configured to be arranged in contact with a patient's bone during an operating configuration of the prosthetic implant; and
a support portion, coupled to the contact and delivery portion, comprising a plurality of ducts, each having at least one inlet section, made in an operative part of the prosthetic implant that can be accessed from the outside by the surgeon in the operating configuration, and at least one outlet section which opens into the contact and delivery portion;
said contact and delivery portion being configured to deliver said medical substance evenly onto said bone when said medical substance is injected through at least one duct of said plurality of ducts during the operating configuration.

2. The prosthetic implant according to claim 1, wherein said ducts have a diameter between 0.5 mm and 4.5 mm, preferably of 2 mm.

3. The prosthetic implant according to claim 1, wherein said contact and delivery portion and said support portion are made in one piece by using an additive manufacturing technique, preferably by Electron Beam Melting (EBM) or Direct Metal Laser Melting (DMLS).

4. The prosthetic implant according to claim 1, wherein said prosthetic implant is made of an alloy that can be selected from:
a titanium alloy, in particular an alloy comprising Titanium, Aluminium and Vanadium according to the formula Ti6Al4V;
an alloy comprising Cobalt, Chromium and Molybdenum.

5. The prosthetic implant according to claim 1, made in the form of a cup wherein:
said contact and delivery portion defines an external hemispherical shell of said cup suitable for engaging an acetabulum of the patient during the operating configuration,
said support portion defines an internal hemispherical shell internally coupled to the external hemispherical shell and having a bottom surface shaped like a circular crown defining said operative part of the prosthetic implant that can be accessed from the outside by the surgeon, on which at least one inlet section of the ducts is arranged.

6. The prosthetic implant according to claim 5, wherein said internal hemispherical shell comprises a containment wall shaped to contain the medical substance when it is injected during the operating configuration, and at least one lightening portion facing said external hemispherical shell so that it is interposed between said external hemispherical shell and said containment wall, said at least one lightening portion having a structure provided with a plurality of interconnected cavities, said plurality of ducts being at least partially embedded in said structure.

7. The prosthetic implant according to claim 6, wherein said internal hemispherical shell comprises one or more ribs extending from the containment wall to the external hemispherical shell so that they divide said at least one lightening portion into two or more sectors, each sector being associated with at least one outlet section.

8. The prosthetic implant according to claim 6, wherein each duct is at least partially, preferably completely, incorporated in the structure of the lightening portions.

9. A kit for delivering a medical substance to an area of contact between a prosthetic implant and a bone of a skeletal structure of a patient, comprising:
a prosthetic implant according to claim 1, which can be installed in contact with a bone of a patient during an operating configuration;
at least one injection device containing a medical substance suitable to be injected through at least one duct of said plurality of ducts during the operating configuration.
